(19) United States
(12) Patent Application Publication
(10) Pub. No.: US 2026/0185055 A1
(43) Pub. Date: Jul. 2, 2026

(54) SYSTEMS AND METHODS OF DIFFERENTIATING STEM CELLS WITHIN BIOREACTOR

(71) Applicant: CORNING INCORPORATED, CORNING, NY (US)

(72) Inventor: Himabindu Nandivada Bailey, Burlington, MA (US)

(21) Appl. No.: 19/130,238

(22) PCT Filed: Nov. 3, 2023
(86) PCT No.: PCT/US2023/036732

§ 371 Date: May 15, 2025

Related U.S. Application Data
(60) Provisional application No. 63/425,339, filed on Nov. 15, 2022.

(51) Int. Cl. C12N 5/074; C12M 1/00; C12M 1/34

(52) U.S. Cl. CPC C12N 5/0696; C12M 23/40; C12M 29/10; C12M 29/18; C12M 41/44; C12M 47/02

(57) ABSTRACT

A method of using a cell culture bioreactor for differentiating stem cells in situ in the bioreactor is provided. The method includes providing a bioreactor vessel that has a cell culture chamber with an inlet for flowing fluid into the cell culture chamber and an outlet for flowing fluid out of the cell culture chamber. The bioreactor vessel also includes a cell substrate disposed in the cell culture chamber for culturing cells thereon. The method includes seeding undifferentiated stem cells onto the cell substrate in the cell culture chamber and perfusing the cell culture chamber with a differentiation media to promote differentiation of the undifferentiated stem cells into a specified cell lineage, whereby the undifferentiated stem cells become differentiated cells.

### CROSS-REFERENCE TO RELATED APPLICATIONS

This application claims the benefit of priority under 35 U.S.C. § 119 of U.S. Provisional Application Ser. No. 63/425,339 filed on Nov. 15, 2022, the content of which is relied upon and incorporated herein by reference in its entirety.

### FIELD OF THE DISCLOSURE

This disclosure general relates to systems and methods for coating of substrates in cell culture bioreactors. In particular, the present disclosure relates to in situ coating of substrates in perfusion bioreactor vessels.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Adherent cell culture is dominating the production of viral vectors for gene and modified cell therapy. This is because cells used for viral vector production are mostly anchorage-dependent. Viral vectors are commonly used to deliver genetic materials into cells and tissues so that genetic defects can be corrected, cellular and tissue function be enhanced, or the production of cellular products be improved, ultimately leading to potential curative treatment. Adherent cell culture is also dominating scale up of stem cells for regenerative medicine. This is because stem cells such as induced pluripotent stem cells (iPSCs) and mesenchymal stem cells (MSCs) are also inherently anchorage-dependent. Stem cells hold great promise for cell therapy, tissue engineering, and regenerative medicine as well as pharmaceutical and biotechnological applications. There is a strong need for reliable and efficient platforms to scale up adherent cell culture.

There are many materials used for cell culture substrates in bioreactors. One of these is polyethylene terephthalate (PET), which is primarily used for viral vector and vaccine production, but its usefulness in other applications for adherent cell culture is limited. For example, many of the existing fixed bed bioreactors have not been used for stem cell culture. Therefore, there is a strong need to develop a fixed bed cell culture substrate with functionalized or coated surfaces that can support the culture of a broader range of cell types.

Mammalian cells are used to produce therapeutic proteins, monoclonal antibodies, viral vectors, and even cultured meat. Furthermore, in tissue engineering and regenerative medicine billions of stem cells are used to fabricate tissue engineered constructs or to replenish lost or damaged cells in degenerative diseases. Although suspension cell culture is widely used to produce proteins and antibodies, adherent cell culture is dominating the production of viral vectors for gene and modified cell therapy, as well as stem cells for regenerative medicine. Viral vectors are commonly used to deliver genetic materials into cells and tissues so that genetic defects can be corrected, cellular and tissue function be enhanced, or the production of cellular products be improved, ultimately leading to potential curative treatment.

Stem cells hold great promise for cell therapy, tissue engineering, and regenerative medicine, as well as pharmaceutical and biotechnological applications. However, cells used for viral vector production are mostly anchorage-dependent; similarly, stem cells such as induced pluripotent stem cells (iPSCs) and mesenchymal stem cells (MSCs) are also inherently anchorage-dependent. Human pluripotent stem cells (hPSCs), which include human embryonic stem cells (hESCs) and human induced pluripotent stem cells (hiPSCs) have the capability for indefinite self-renewal and can differentiate into all derivatives of the three primary germ layers. These cells have tremendous potential in clinical applications, regenerative medicine, tissue engineering, drug screening and study of early developmental biology. There is a need for large number of hPSCs for cell therapy and regenerative medicine applications. This requires technologies that enable efficient scale up and expansion of hPSCs. Further, for cell therapy and regenerative medicine applications, there is a need to produce large number of cells differentiated into specific lineages. This requires technologies that can support the efficient and effective differentiation of hPSCs.

Culture of hPSCs requires controlled culture environments to ensure attachment, survival, proliferation, self-renewal, and maintenance of pluripotency and genomic stability of the cells. Human PSCs are cultured as on naturally-derived extracellular matrices (e.g., Corning® Matrigel® matrix, Life Technologies'Geltrex®), recombinant proteins (such as Vitronectin, Laminin-511, Laminin-521), and synthetic surface (Corning® Synthemax®).

There is a need for cell culture bioreactor systems, and methods of using such systems, that allow not only culturing of adult cells that terminally differentiated, but also differentiation of hPSCs in the bioreactor.

### SUMMARY

According to embodiments, a method of differentiating cells in a bioreactor system is provided. The method includes seeding stem cells in a bioreactor having a cell culture chamber containing a cell substrate, and culturing the stem cells using cell culture media that support undifferentiated stem cell culture. The method further includes perfusing the cell culture chamber with a differentiation media to promote differentiation of the stem cells into one or more lineages. The method further includes, after differentiation is complete, washing the cells with a washing solution. The washing solution can be a fluid media and/or a phosphate-buffered solution (PBS). The method can further include additional culturing of the differentiated cells in the bioreactor. Optionally, the method also includes harvesting the differentiated cells or a cell by-product. Harvesting can include perfusing the cell culture chamber wit ha dissociation reagent to release and remove the differentiated cells from the bioreactor. Removal of the differentiated cells from the bioreactor can also include pressurizing the bioreactor to force the differentiated cells and any media out of the bioreactor.

According to embodiments, a method of coating a cell culture substrate in situ in a bioreactor is provided. The method includes providing a bioreactor vessel having a cell culture chamber within the bioreactor vessel. The cell culture chamber includes an inlet for flowing fluid into the cell culture chamber and an outlet for flowing fluid out of the cell culture chamber. The bioreactor vessel also includes a cell substrate disposed in the cell culture chamber for culturing cells thereon. The method incudes providing a coating solution for coating the cell substrate; inputting the coating solution into the cell culture chamber through the inlet such that the coating solution contacts the cell substrate to coat the cell substrate; and removing an excess of the coating solution from the cell culture chamber via the outlet or the inlet. After removing the coating solution, a coated cell substrate remains in the cell culture chamber.

According to embodiments, a method of culturing cells in a bioreactor is provided. The method includes coating a cell substrate within the bioreactor as described herein; seeding cells on the coated cell substrate; culturing the cells on the coated cell substrate; and harvesting a product of the culturing of the cells.

According to embodiments, a system of culturing adherent cells in a bioreactor is provided. The system includes a bioreactor vessel having a cell culture chamber within the bioreactor vessel. The cell culture chamber includes an inlet for flowing fluid into the cell culture chamber and an outlet for flowing fluid out of the cell culture chamber. The cell culture substrate also includes a cell substrate disposed in the cell culture chamber for culturing cells thereon. A recirculation loop is also provided that can supply fluid to the bioreactor vessel via the inlet and remove fluid from the bioreactor vessel via the outlet. The system further includes a coating solution vessel fluidly connected to the cell culture chamber for holding a substrate coating solution.

### BRIEF DESCRIPTION OF THE DRAWINGS

FIG. 1 is a schematic drawing of a fixed bed bioreactor system for coating a cell substrate in the bioreactor, according to embodiments.

FIG. 2 is a schematic drawing of the bioreactor system of FIG. 1 during a step of coating the cell substrate, according to embodiments.

FIG. 3 is a schematic drawing of the bioreactor system of FIGS. 1 and 2 after coating the cell substrate, according to embodiments.

FIG. 4 is a schematic representation of a cell culture system, according to one or more embodiments.

FIG. 5 shows a process flow chart for coating a cell substrate in a bioreactor for culturing cells, according to one or more embodiments.

FIG. 6 is a schematic representation of a cell culture system for differentiating stem cells and culturing differentiated cells, according to one or more embodiments.

FIG. 7 shows a process flow chart for seeding, differentiating, and culturing stem cells in a bioreactor, according to one or more embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

The surface chemistry of adherent cell culture substrates may need to be modified to provide desired cell adhesion properties. Such modifications can be made through the chemical treatment of the polymer material of the substrate or by grafting cell adhesion molecules to the substrate surface. Alternatively, substrates can be coated with thin layer of biocompatible hydrogels that demonstrate cell adherence properties, including, for example, collagen or Matrigel®. A variety of coatings can be used, including extracellular matrix proteins, fibronectin, collagen, a hydrogel solution, a polymer solution, and recombinant proteins, for example. Any suitable coating can be used, as would be understood by a person of skill in the art. Alternatively, surfaces of the cell substrate can be rendered with cell adhesive properties through the treatment processes with various types of plasmas, process gases, and/or chemicals known in the industry. In one or more embodiments, however, the cell substrate is capable of providing an efficient cell growth surface without surface treatment. For the simplification and flexibility in manufacturing of bioreactors, it may be advantageous to provide cell culture bioreactors that are uncoated so that end users, depending on their cell type or application of interest, can decide what type of coating to apply to the cell substrate in the bioreactor.

Therefore, embodiments provided herein conveniently allow users to coat the substrate in situ inside the bioreactor. Thus, by provided a bioreactor pre-packed with the cell substrate and enabling the cell substrate to be coated in place, a solution is provided that is flexible and easy to use, while minimizing risks of contamination or assembly error that may arise if the end user had to remove the substrate and re-pack it into the bioreactor after coating.

To provide users additional flexibility, embodiments of this disclosure include systems and methods to enable differentiation of stem cells in situ in the bioreactor vessel. This allows bioreactor systems of this disclosure to be used with undifferentiated cells (e.g., stem cells) that can be tailored to a desired cell lineage and application within the bioreactor itself.

Embodiments of this disclosure relate to systems and methods for in situ coating of cell substrates within fixed bed bioreactors, and systems and methods for culturing cells within such bioreactors. These fixed bed bioreactors can be used for seeding, culturing, and/or expanding cells of various types, including mesenchymal stem cells, cancer cells, T-cells, fibroblasts, and myoblasts, among others. Before culturing adherent cells on the cell substrate, it may be desirable to coat the cell substrate with a coating to improve performance for a particular cell type or application. For example, an adherence-promoting compound may be applied to a cell growth surface to promote contact, e.g., adherence and subsequent expansion of cells, such as a human mesenchymal stem cells (hMSCs).

Embodiments of this disclosure include fixed bed bioreactor systems for cell culture. According to aspects of such embodiments, the bioreactor system may be a closed system in which contents of the bioreactor system are not directly exposed to the atmosphere to prevent contamination. The bioreactor system may be automated. In aspects of embodiments, the system may include cell media and/or coating solution for coating a cell substrate within the bioreactor. The bioreactor system may include a fluid flow path that includes a path from an inlet of the bioreactor vessel, through a cell culture chamber containing a substrate, and out of the bioreactor via an outlet. The fluid flow path may also include one or more media conditioning vessels fluidly connected to the cell culture chamber and that may be integrated with or separate from the bioreactor vessel. The system may further include a coating solution source fluidly connected to the fluid flow path and configured to inject a coating solution into the fluid flow path such that the coating solution runs into the cell culture space. The system may include one or more pumps for circulating media through the fluid flow path and/or for injecting the coating solution into the cell culture space.

In embodiments, the system includes a controller for controlling operation of the system, including the one or more pumps. The controller may include a computer system including a processor. The controller is configured, in embodiments, to control the one or more pumps to circulate a fluid (e.g., cell culture media or coating solution) at a flow rate through the fluid flow path. The controller, according to aspects of embodiments, controls the one or more pumps to transfer cell culture media, nutrients, and/or cells from a source to the fluid flow path and into the bioreactor. The system may include a graphical user interface and a memory, in communication with and readable by the processor, and containing instructions. When instructions are executed by the processor, the processor receives an instruction to coat the cell substrate in the bioreactor, for example. In response to the instruction to coat the bioreactor, the processor may execute a series of steps to coat the bioreactor and may next receive an instruction to load cells into the bioreactor, for example. In response to instructions to load cells, the processor may execute a series of steps to load the cells from a cell inlet source, for example, into the bioreactor.

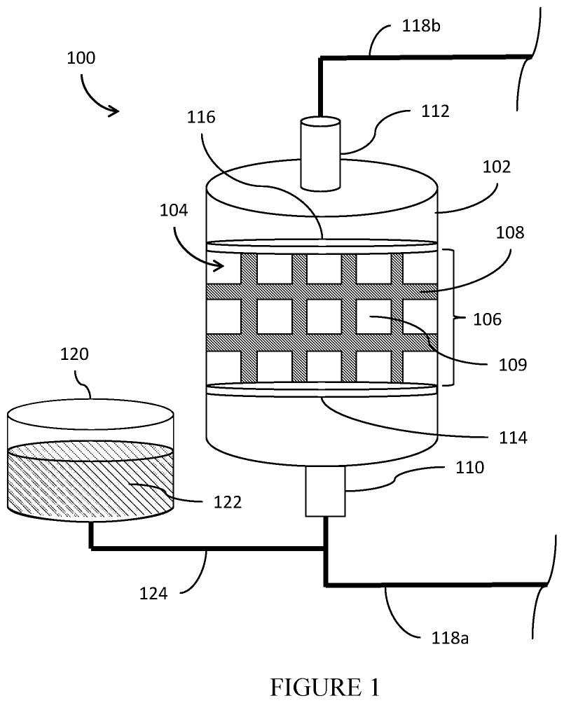

FIG. 1 shows a cell culture system 100 according to embodiments of this disclosure. The cell culture system 100 includes a cell culture vessel 102 having an interior reservoir containing a cell culture space 104 in which adherent cells can be seeded, cultured, transfected, differentiated, and/or harvested. A fixed bed cell substrate 106 is disposed in the cell culture space 104. The fixed bed 106 is made of a cell substrate 108, as described herein. The cell substrate 108, according to aspects of embodiments, is a porous material having a predetermined structure, such as an ordered array of openings 109 or pores and rigid filaments. The structure and arrangement of the cell substrate 108 and fixed bed 106 are such that the fixed bed 106 enables uniform fluid flow therethrough, which improves cell seeding, nutrient distribution, substrate coating, and cell harvesting. The cell culture system 100 may include an inlet distribution plate 114 and outlet distribution plate 116 to help distribute media, cells, coating solution, and other fluids evenly throughout the fixed bed. The cell culture system 100 also includes an inlet 110 and an outlet 112 for flowing fluid into and out of, respectively, the cell culture space 104. The inlet 110 and outlet 112 are connected to a fluid flow path 118a, 118b for supplying a fluid flow pathway into and out of the cell culture vessel 102. The fluid flow path 118a, 118b may form a perfusion flow path that recirculates fluid in a loop through the bioreactor, as discussed further below.

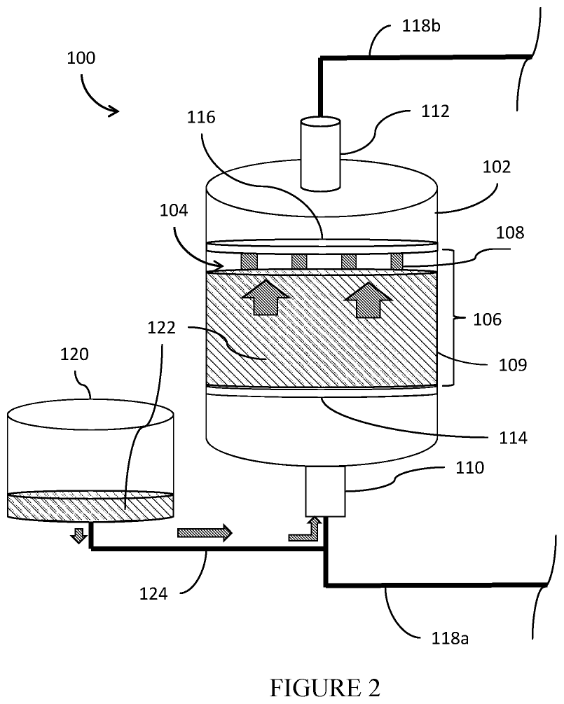

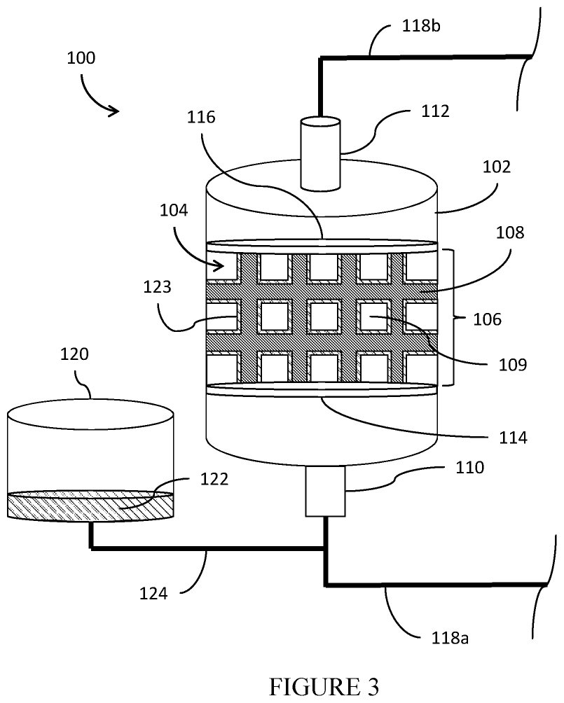

According to embodiments, the cell culture system 100 includes a coating solution vessel 120 fluidly connected to the cell culture vessel 102 and able to supply a coating solution 122 contained therein to the cell culture space 104. The coating solution vessel 120 may be connected via a coating pathway 124 to the fluid flow path 118a or 118b, or may be directly connected to the cell culture vessel 102 via a separate inlet outside of the fluid flow path 118a, 118b. As shown in FIG. 2, the cell culture system 100 can supply the coating solution 112 from the coating solution vessel 120 to the cell culture space 104, which contains the cell substrate 108. After the coating solution 122 fills the cell culture space 104 and contacts the cell substrate 108, the coating solution 122 may be left in contact with the cell substrate 108 for a predetermined time, or until application of the coating to the cell substrate 108 is completed (e.g., by some physical or chemical reaction; by some external stimulus such as heating, cooling, or radiation). After coating is deemed to be completed, the coating solution 122 can be removed from the cell culture space 104, as shown in FIG. 3, leaving behind the coated cell substrate made of a coating 123 on the cell substrate 108. Removal of the coating solution 122 from the cell culture space can be accompanied by a wash with a washing solution (not pictured), either while the excess coating solution is being removed or sometime after the removal is completed. As a result, a bioreactor with a cell substrate suited to a desired cell culture application is provided.

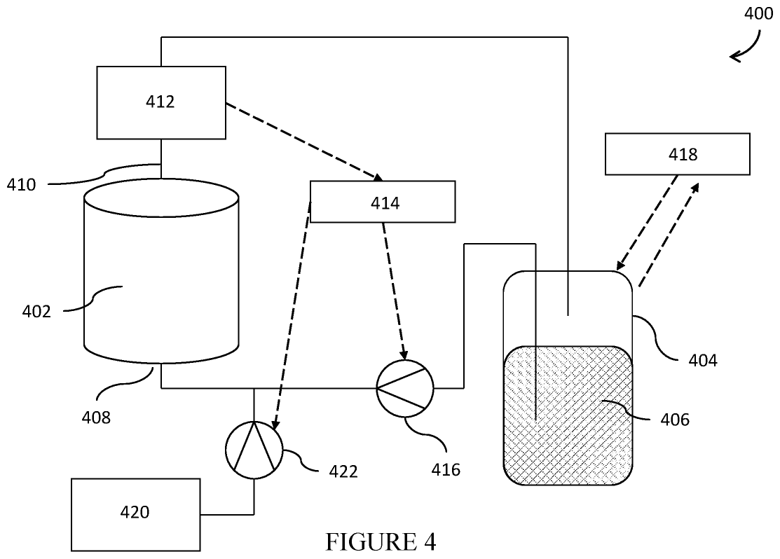

FIG. 4 shows a cell culture system 400 according to one or more embodiments. The system 400 includes a bioreactor 402 housing the fixed bed cell substrate of one or more embodiments disclosed herein. The bioreactor 402 can be fluidly connected to a media conditioning vessel 404, and the system is capable of supplying a cell culture media 406 within the conditioning vessel 404 to the bioreactor 402. The media conditioning vessel 404 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 404 also contains an impeller for media mixing. All media parameters measured by sensors listed above can be controlled by a media conditioning control unit 418 in communication with the media conditioning vessel 404, and capable of measuring and/or adjusting the conditions of the cell culture media 406 to the desired levels. As shown in FIG. 4, the media conditioning vessel 404 is provided as a vessel that is separate from the bioreactor vessel 402. This can have advantages in terms of being able to condition the media separate from where the cells are cultured, and then supplying the conditioned media to the cell culture space. However, in some embodiments, media conditioning can be performed within the bioreactor vessel 402.

The media from the media 406 conditioning vessel 404 is delivered to the bioreactor 402 via an inlet 408, which may also include an injection port for cell inoculum to seed and begin culturing of cells. The bioreactor vessel 402 may also include on or more outlets 410 through which the cell culture media 406 exits the vessel 402. In addition, cells or cell products may be output through the outlet 410. To analyze the contents of the outflow from the bioreactor 402, one or more sensors 412 may be provided in the line. In some embodiments, the system 400 includes a flow control unit 414 for controlling the flow into the bioreactor 402. For example, the flow control unit 414 may receive a signal from the one or more sensors 412 (e.g., an O₂ sensor) and, based on the signal, adjust the flow into the bioreactor 402 by sending a signal to a pump 416 (e.g., peristaltic pump) upstream of the inlet 408 to the bioreactor 402. Thus, based on one or a combination of factors measured by the sensors 412, the pump 416 can control the flow into the bioreactor 402 to obtain the desired cell culturing conditions.

The media perfusion rate is controlled by the signal processing unit 414 that collects and compares sensors signals from media conditioning vessel 404 and sensors located at the fixed bed bioreactor outlet 410. Because of the pack flow nature of media perfusion through the fixed bed bioreactor 402, nutrients, pH and oxygen gradients are developed along the fixed bed. The perfusion flow rate of the bioreactor can be automatically controlled by the flow control unit 414 operably connected to the peristaltic pump 416. Examples of additional controls and system components can be found in U.S. Patent Application Publication No. US2020/0248124A1, which is incorporated herein by reference.

The cell culture system 400 also includes a coating solution vessel 420 fluidly connected to the bioreactor vessel 402 and able to supply a coating solution contained therein to the bioreactor vessel 402. The coating solution is chosen to improve the performance of a given cell culture application. A pump 422 can supply the coating solution directly to the bioreactor vessel 402 or to a fluid flow pathway connected to the bioreactor vessel 402. The controller 414 can send a signal to activate the pump 422 when it is time to supply the coating solution to the cell substrate in the bioreactor. After the coating solution fills the bioreactor system 402 and contacts the cell substrate, the coating solution may be left in contact with the cell substrate for a predetermined time, or until application of the coating to the cell substrate is completed (e.g., by some physical or chemical reaction; by some external stimulus such as heating, cooling, or radiation). After coating is deemed to be completed, the coating solution can be removed from the cell culture space. For example, the controller 414 can send a signal to a pump to remove the excess coating solution and/or to supply a washing solution to the reactor 402 to assist in removing the excess coating solution. The washing solution can contain a media or a fluid, such as phosphate buffered saline (PBS) or other cell culture media.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the substrate. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture substrate made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture substrate having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a fixed bed substrate is provided with a structurally defined surface area for adherent cells to attach and proliferate that has good mechanical strength and forms a highly uniform multiplicity of interconnected fluidic networks when assembled in a fixed bed or other bioreactor. In particular embodiments, a mechanically stable, non-degradable woven mesh can be used as the substrate to support adherent cell production. The cell substrate disclosed herein supports attachment and proliferation of anchorage dependent cells in a high volumetric density format. Uniform cell seeding of such a substrate is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed substrate, and can avoid formation of large and/or uncontrollable 3D cellular aggregates with limited nutrient diffusion and increased metabolite concentrations. Thus, the substrate eliminates diffusional limitations during operation of the bioreactor. In addition, the substrate enables easy and efficient cell harvest from the bioreactor. The structurally defined substrate of one or more embodiments enables complete cell recovery and consistent cell harvesting from the fixed bed of the bioreactor.

According to some embodiments, a method of cell culturing is also provided using bioreactors with the substrate for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the fixed bed. This construction enables improved cell seeding, nutrient delivery, cell growth, and cell harvesting. According to one or more particular embodiments, the substrate is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the substrate has a high surface-to-volume ratio for culturing anchorage dependent cells. According to various embodiments, the substrate can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

According to embodiments, a cell culture substrate can be a woven mesh layer made of a first plurality of fibers running in a first direction and a second plurality of fibers running in a second direction. The woven fibers of the substrate form a plurality of openings, which can be defined by one or more widths or diameters. The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Without wishing to be bound by theory, it is believed that the three-dimensional structure of the substrate is advantageous as it provides a large surface area for culturing adherent cells, and the structural rigidity of the mesh can provide a consistent and predictable cell culture substrate structure that enables uniform fluid flow.

In one or more embodiments, a fiber may have a diameter in a range of about 10 μm to about 1000 μm; about 100 μm to about 750 μm; about 125 μm to about 600 μm; about 150 μm to about 500 μm; about 200 μm to about 400 μm; about 200 μm to about 300 μm; about 10 μm to about 300 μm; about 20 μm to about 250 μm; about 20 μm to about 170 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 10 μm×10 μm to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; about 10 μm to about 200 μm; about 20 μm to about 150 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture substrate comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture substrate includes a stack, roll, or other arrangement of overlapping substrate, the packing density of the cell culture substrate will impact the surface area of the fixed bed substrate. Packing density can vary with the packing thickness of the substrate material (e.g., the space needed for a layer of the substrate). For example, if a stack of cell culture substrate has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture substrate with a lower density packing of layers (e.g., when higher permeability is a priority) or a higher density of packing (e.g., when maximizing substrate surface area is a priority). According to one or more embodiments, the packing thickness can be from about 10 μm to about 1000 μm; about 100 μm to about 750 μm; about 125 μm to about 600 μm; about 150 μm to about 500 μm; about 200 μm to about 400 μm; about 200 μm to about 300 μm; about 10 μm to about 300 μm; or about 20 μm to about 250 μm.

The above structural factors can determine the surface area of a cell culture substrate, whether of a single layer of cell culture substrate or of a cell culture substrate having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture substrate can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

By using a structurally defined culture substrate of sufficient rigidity, high-flow-resistance uniformity across the substrate or fixed bed is achieved. According to various embodiments, the substrate can be deployed in monolayer or multilayer formats. This flexibility eliminates diffusional limitations and provides uniform delivery of nutrients and oxygen to cells attached to the substrate. In addition, the open substrate lacks any cell entrapment regions in the fixed bed configuration, allowing for complete cell harvest with high viability at the end of culturing. The substrate also delivers packaging uniformity for the fixed bed, and enables direct scalability from process development units to large-scale industrial bioprocessing unit. The ability to directly harvest cells from the fixed bed eliminates the need of resuspending a substrate in a stirred or mechanically shaken vessel, which would add complexity and can inflict harmful shear stresses on the cells. Further, the high packing density of the cell culture substrate yields high bioprocess productivity in volumes manageable at the industrial scale.

Embodiments of this disclosure include a cell substrate that is a multilayer substrate. The multilayer substrate includes a first mesh substrate layer and a second mesh substrate layer stacked on top of each other. The number of substrates in the stack can be adjusted to meet with the required density or number of cells (or cell product). However, embodiments are not limited to this configuration, and various configurations can be used for the cell substrate. For example, the cell substrate can be a roll of cell substrate material, or small pieces of substrate material fixed into the reactor.

The geometry of the mesh substrate layers is designed to allow efficient and uniform flow through one or multiple substrate layers. In addition, the structure of the cell substrate can accommodate fluid flow through the substrate in multiple orientations. For example, the direction of bulk fluid flow can be perpendicular to the major side surfaces of the first and second substrate layers, or the cell substrate can be oriented with respect to the flow such that the sides of the substrate layers are parallel to the bulk flow direction. In addition to fluid flow being perpendicular or parallel to the first and second sides of the mesh layers, the substrate can be arranged with multiple pieces of substrate at intermediate angles, or even in random arrangements with respect to fluid flow. This flexibility in orientation is enabled by the essentially isotropic flow behavior of the woven substrate. In contrast, substrates for adherent cells in existing bioreactors do not exhibit this behavior and instead their fixed beds tend to create preferential flow channels and have substrate materials with anisotropic permeability. The flexibility of the cell substrate of the current disclosure allows for its use in various applications and bioreactor or container designs while enabling better and more uniform permeability throughout the bioreactor vessel.

As discussed herein, the cell substrate can be used within a bioreactor vessel, according to one or more embodiments. For example, the substrate can be used in a fixed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

A cell culture system is provided, according to one or more embodiments, in which the cell culture substrate is used within a culture chamber of a bioreactor vessel. Within the cell culture chamber is a fixed bed cell substrate that is made from a stack of cell substrate layers. The cell substrate layers are stacked with the first or second side of a substrate layer facing a first or second side of an adjacent substrate layer. The bioreactor vessel has an inlet at one end for the input of media, cells, and/or nutrients into the culture chamber, and an outlet at the opposite end for removing media, cells, or cell products from the culture chamber. By allowing stacking of substrate layers in this way, the system can be easily scaled up without negative impacts on cell attachment and proliferation, due to the defined structure and efficient fluid flow through the stacked substrates. While the vessel may generally be described as having an inlet and an outlet, some embodiments may use one or both of the inlet and outlet for flowing media, cells, or other contents both into and out of the culture chamber. For example, inlet may be used for flowing media or cells into the culture chamber during cell seeding, perfusion, or culturing phases, but may also be used for removing one or more of media, cells, or cell products through the inlet in a harvesting phase. Thus, the terms “inlet” and “outlet” are not intended to restrict the function of those openings.

In one or more embodiments, flow resistance and volumetric density of the fixed bed can be controlled by interleaving substrate layers of different geometries. In particular, mesh size and geometry (e.g., fiber diameter, opening diameter, and/or opening geometry) define the fluid flow resistance in fixed bed format. By interlaying meshes of different sizes and geometries, flow resistance can be controlled or varied in one or more specific portions of the bioreactor. This will enable better uniformity of liquid perfusion in the fixed bed. Various combinations of meshes of different sizes are possible to obtain different profiles of volumetric density of cells growth surface and flow resistance. For example, a fixed bed with zones of varying volumetric cells densities (e.g., a series of zones creating a pattern of low/high/low/high, etc. densities) can be assembled by interleaving meshes of different sizes.

According to embodiments, the bulk flow direction of fluid through the bioreactor is in a direction from the inlet to the outlet, and, in aspects of embodiments, the first and second major sides of the cell substrate layers are perpendicular to the bulk flow direction. In contrast, aspects of some embodiments include a bioreactor vessel and stack of cell substrates within the culture space that have first and second sides that are parallel to a bulk flow direction. Thus, the cell substrates of embodiments of this disclosure can be employed in either configuration. In either of these examples, the cell substrates are sized and shaped to fill the interior space defined by the culture chamber so that the culture spaces in each vessel are filled for cell growth surfaces to maximize efficiency in terms of cells per unit volume. The cell culture space of the system may be fed by a single inlet and have a single outlet, or may have multiple inlets and/or multiple outlets. However, according to various embodiments herein, distribution plates can be used to help distribute the media, cells, or nutrients across a cross-section of the fixed bed and thus improve uniformity of fluid flow through the fixed bed. As such, the multiple inlets represent how a distribution plate can be provided with a plurality of holes across the fixed-bed cross-section for creating more uniform flow.

In some embodiments, the fixed bed is arranged such that the cell substrate is formed into a cylindrical roll. For example, a sheet of a cell substrate material (e.g., one or more mesh substrates) is rolled into a cylinder about a central longitudinal axis of the cell culture space. The cylindrical roll has a width along a dimension perpendicular to the central longitudinal axis and a height along a direction parallel to the central longitudinal axis. In one or more embodiments, the cylindrical roll is designed to be within a bioreactor vessel such that the central longitudinal axis is parallel to a direction of bulk flow of fluid through the bioreactor or culture chamber that houses the cylindrical roll. The bioreactor system may further include a central support member around which the cell substrate is positioned. The central support member can be provided purely for physical support and/or alignment of the cell substrate, but can also provide other functions, according to some embodiments. For example, the central support member can be provided with one or more openings for supplying media to the cell substrate along the length of the fixed bed. In other embodiments, the central support member may include one or more attachment sites for holding one or more portions of the cell culture substrate at the inner part of the cylindrical roll. These attachment sites may be hooks, clasps, posts, clamps, or other means of attaching the mesh sheet to the central support member.

One or more embodiments of this disclosure offer a cell inoculation step that is different from conventional methods. In conventional methods, a pack bed with a conventional substrate is filled with culture media and concentrated inoculum is injected into the media circulation loop. The cell suspension is pumped through the bioreactor at increased flow rate to reduce nonuniformity of cell seeding via capture on the conventional fixed bed substrate. In such conventional methods, the pumping of cells in the circulation loop at an elevated flow rate continues for perhaps several hours until the majority of the cells are captured in packed bed bioreactor. However, because of the nonuniform deep bed filtration nature of conventional packed bed bioreactors, cells are distributed nonuniformly inside the packed bed with the higher cell density at the inlet region of the bioreactor and lower cell density at the outlet region of the bioreactor.

In contrast, according to embodiments of the present disclosure, cell inoculum of equal volume to the void volume of the culture chamber in the bioreactor is directly injected into the packed bed through a cell inoculum injection port at the inlet 408 of the bioreactor 402 (FIG. 4). The cell suspension is then uniformly distributed inside the packed bed because of uniform and continuous fluidic passages present in the cell culture substrate described herein. To prevent cells sedimentation due to gravity forces at the initial seeding stage, media perfusion can be started immediately after the inoculum injection. The perfusion flow rate is maintained below a preprogrammed threshold to balance the force of gravity and to avoid cells being washed from the packed bed bioreactor. Thus, at the initial cell attachment stage, cells are gently tumbled inside the packed bed and uniform cells distribution and attachment on available substrate surface is achieved.

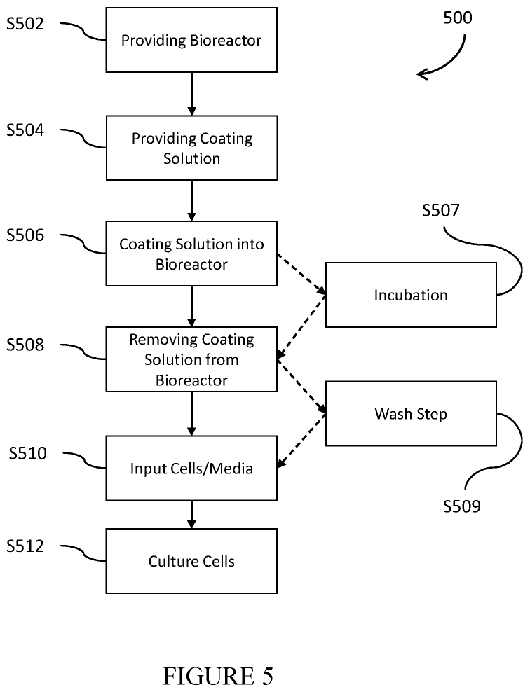

Embodiments include methods of coating a cell substrate in situ within a bioreactor vessel. For example, the system 400 of FIG. 4 can be operated according to process steps according to one or more embodiments. As shown in FIG. 5, this method 500 can include providing a cell culture bioreactor (S502), providing a coating solution (S504), and flowing the coating solution into the cell culture space of the bioreactor (S506). Following insertion of the coating solution into the cell culture space S506, an incubation step S507 can follow, during which the coating solution remains in the cell culture space to coat the substrate. This incubation step S507 can include the addition of some additional stimulus, such as heating, cooling, or applying some kind of radiation, to assist in the coating solution forming a coating on the cell substrate. During the incubation step S507, the coating solution can sit stagnant in the cell culture space or can be perfused through the cell culture space, either in a loop or a one-time perfusion through the reactor. The incubation period S507 can also include a reaction occurring to form the coating on the cell substrate from the coating solution. The method 500 can further include a step (S508) of removing the coating solution from the bioreactor after a coating has been formed on the cell substrate. The step of removing the coating solution S508 can occur after a predetermined time period deemed sufficient for coating given the process parameters. Optionally, a washing step S509 can be performed during or after the removing step S508. For example, the coating solution can be forced out of the bioreactor by the injection of a washing solution that flushes the bioreactor, or the washing solution can be input into the bioreactor after the excess coating solution has been removed. Next, the cell culture process can proceed, including seeding and attaching cells (S510), as well as cell culture media, followed by a culturing process that may include cell expansion, transfection, cell differentiation, production of viral vector or other cell products, and harvesting.

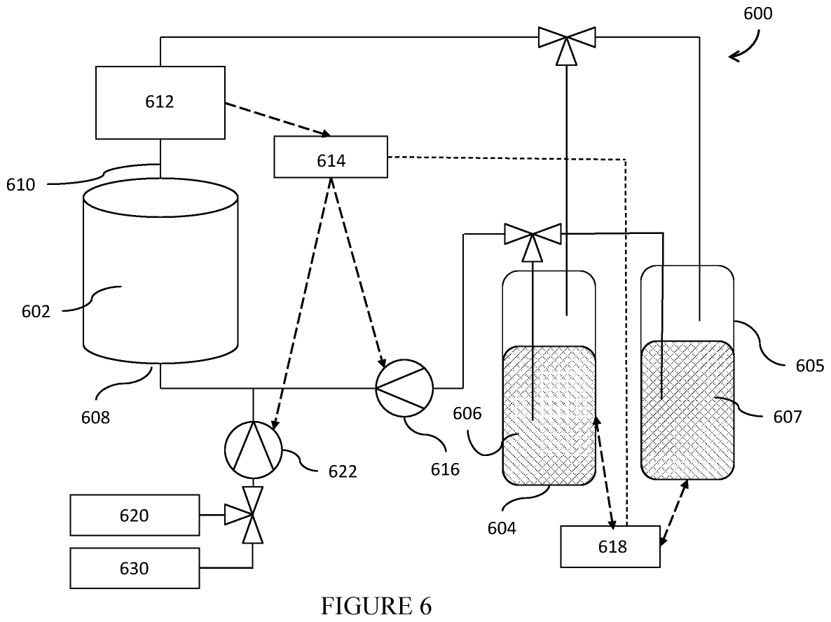

FIG. 6 shows a cell culture system 600 according to one or more embodiments. The system 600 includes a bioreactor 602 housing the fixed bed cell substrate of one or more embodiments disclosed herein. The bioreactor 602 can be fluidly connected to a media conditioning vessel 604, and the system is capable of supplying a cell culture media 606 within the conditioning vessel 604 to the bioreactor 602. The media 606 of the media conditioning vessel can be a differentiation media for feeding to undifferentiated cells at the beginning of a cell culture process, according to embodiments. After differentiation, the media 606 can be replaced with a media suitable for culturing the differentiated cells. Alternatively, as shown in FIG. 6, the system 600 can have two media conditioning vessels 604 and 605: one for supplying a differentiation media 607 and one for supplying a regular cell culture media 606 for after differentiation. The media conditioning vessel 604 and/or 605 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 604 and/or 605 also contains an impeller for media mixing. All media parameters measured by sensors listed above can be controlled by a media conditioning control unit 618 in communication with the media conditioning vessel 604 and/or 605, and capable of measuring and/or adjusting the conditions of the cell culture media 606 and/or differentiation media 607 to the desired levels. As shown in FIG. 6, the media conditioning vessels 604 and 605 are provided as a vessel that is separate from the bioreactor vessel 602. This can have advantages in terms of being able to condition the media separate from where the cells are cultured, and then supplying the conditioned media to the cell culture space. However, in some embodiments, media conditioning can be performed within the bioreactor vessel 602.

The media 606 and 607 from the media conditioning vessels 604 and 605 is delivered to the bioreactor 602 via an inlet 608, which may also include an injection port for cell inoculum to seed and begin culturing of cells. The bioreactor vessel 602 may also include on or more outlets 610 through which the cell culture media 606 and 607 exit the vessel 602. In addition, cells or cell products may be output through the outlet 610. To analyze the contents of the outflow from the bioreactor 602, one or more sensors 612 may be provided in the line. In some embodiments, the system 600 includes a flow control unit 614 for controlling the flow into the bioreactor 602. For example, the flow control unit 614 may receive a signal from the one or more sensors 612 (e.g., an O₂ sensor) and, based on the signal, adjust the flow into the bioreactor 602 by sending a signal to a pump 616 (e.g., peristaltic pump) upstream of the inlet 608 to the bioreactor 602. Thus, based on one or a combination of factors measured by the sensors 612, the pump 616 can control the flow into the bioreactor 602 to obtain the desired cell culturing conditions.

The media perfusion rate is controlled by the signal processing unit 614 that collects and compares sensors signals from media conditioning vessel 604 and sensors located at the fixed bed bioreactor outlet 610. Because of the pack flow nature of media perfusion through the fixed bed bioreactor 602, nutrients, pH and oxygen gradients are developed along the fixed bed. The perfusion flow rate of the bioreactor can be automatically controlled by the flow control unit 614 operably connected to the peristaltic pump 616. Examples of additional controls and system components can be found in U.S. Patent Application Publication No. US2020/0248124A1, which is incorporated herein by reference.

The cell culture system 600 also includes a coating solution vessel 620 fluidly connected to the bioreactor vessel 602 and able to supply a coating solution contained therein to the bioreactor vessel 602. The coating solution is chosen to improve the performance of a given cell culture application. A pump 622 can supply the coating solution directly to the bioreactor vessel 602 or to a fluid flow pathway connected to the bioreactor vessel 602. The controller 614 can send a signal to activate the pump 622 when it is time to supply the coating solution to the cell substrate in the bioreactor. After the coating solution fills the bioreactor system 602 and contacts the cell substrate, the coating solution may be left in contact with the cell substrate for a predetermined time, or until application of the coating to the cell substrate is completed (e.g., by some physical or chemical reaction; by some external stimulus such as heating, cooling, or radiation). After coating is deemed to be completed, the coating solution can be removed from the cell culture space. For example, the controller 614 can send a signal to a pump to remove the excess coating solution and/or to supply a washing solution to the reactor 602 to assist in removing the excess coating solution. The washing solution can contain a media or a fluid, such as phosphate buffered saline (PBS) or other cell culture media.

The system 600 further includes a cell inoculum 630. According to embodiments, the cell inoculum contains undifferentiated stem cells, which are injected into the bioreactor vessel 602 via the inlet 608. Following injection of the undifferentiated cells, a differentiation media 607 can be perfused into/through the bioreactor 602 to support differentiation of the stem cells within the bioreactor into a desired, differentiated cell line. Following differentiation of the cells, a washing step using a washing solution can be performed. Finally, the differentiated cells can be further cultured using cell culture media 606.

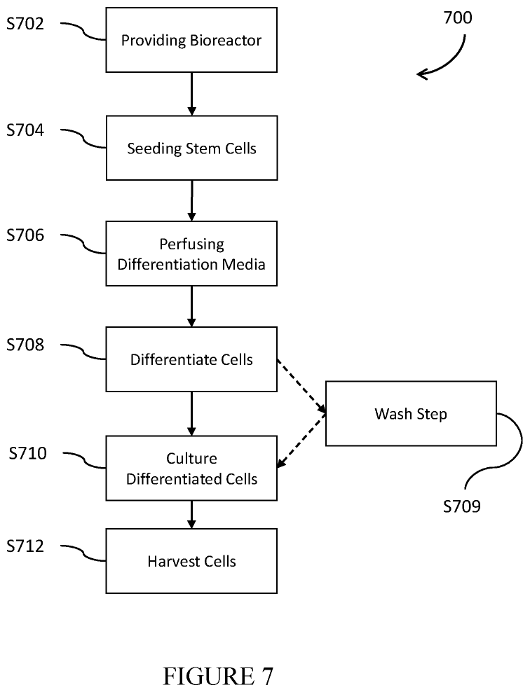

Embodiments include methods of using a bioreactor system for differentiating stem cells within the bioreactor in situ. For example, the system 600 of FIG. 6 can be operated according to process steps according to one or more embodiments. As shown in FIG. 7, this method 700 can include providing a cell culture bioreactor (S702), seeding undifferentiated stem cells onto or into a cell substrate within a cell culture space of the bioreactor (S704), and flowing a differentiation media into the cell culture space of the bioreactor (S706). Following insertion of the differentiation media into the cell culture space, the cells can be differentiated into a desired cell line (S708). Optionally, a washing step S709 can be performed during or after the differentiation step S708. For example, the differentiation media and any waste products can be forced out of the bioreactor by the injection of a washing solution that flushes the bioreactor, or the washing solution can be input into the bioreactor after the excess differentiation media and waste has been removed. Next, the cell culture process of the differentiated cells can proceed (S710). Finally, a harvesting step S712 can remove the differentiated cells and/or cell by-products for further downstream processing.

The cell culture substrate can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture substrate includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture substrate and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture substrate during the culturing of cells, and/or during the inflow or outflow of culture media to the culture chamber.

In one or more embodiments, the cell culture substrate is secured within the culture chamber by a fixing mechanism. The fixing mechanism may secure a portion of the cell culture substrate to a wall of the culture chamber that surrounds the substrate, or to a chamber wall at one end of the culture chamber. In some embodiments, the fixing mechanism adheres a portion of the cell culture substrate to a member running through the culture chamber, such as member running parallel to the longitudinal axis of the culture chamber, or to a member running perpendicular to the longitudinal axis. However, in one or more other embodiments, the cell culture substrate may be contained within the culture chamber without being fixedly attached to the wall of the chamber or bioreactor vessel. For example, the substrate may be contained by the boundaries of the culture chamber or other structural members within the chamber such that the substrate is held within a predetermined area of the bioreactor vessel without the substrate being fixedly secured to those boundaries or structural members.

One aspect of some embodiments provides a bioreactor vessel in a roller bottle configuration. The culture chamber is capable of containing a cell culture substrate and substrate according to one or more of the embodiments described in this disclosure. In the roller bottle configuration, the bioreactor vessel may be operably attached to a means for moving the bioreactor vessel about a central longitudinal axis of the vessel. For example, the bioreactor vessel may be rotated about the central longitudinal axis. The rotation may be continuous (e.g., continuing in one direction) or discontinuous (e.g., an intermittent rotation in a single direction or alternating directions, or oscillating in back and forth rotational directions). In operation, the rotation of the bioreactor vessel causes movement of cells and/or fluid within the chamber. This movement can be considered relative with respect to the walls of the chamber. For example, as the bioreactor vessel rotates about its central longitudinal axis, gravity may cause the fluid, culture media, and/or unadhered cells to remain toward a lower portion of the chamber. However, in one or more embodiments, the cell culture substrate is essentially fixed with respect to the vessel, and thus rotates with the vessel. In one or more other embodiments, the cell culture substrate can be unattached and free to move to a desired degree relative to the vessel as the vessel rotates. The cells may adhere to the cell culture substrate, while the movement of the vessel allows the cells to receive exposure to both the cell culture media or liquid, and to oxygen or other gases within the culture chamber.

By using a cell culture substrate according to embodiments of this disclosure, such as a substrate including a woven or mesh substrate, the roller bottle vessel is provided with an increased surface area available for adherent cells to attach, proliferate, and function. In particular, using a substrate of a woven mesh of monofilament polymer material within the roller bottle, the surface area may increase by of about 2.4 to about 4.8 times, or to about 10 times that of a standard roller bottle. As discussed herein, each monofilament strand of the mesh substrate is capable of presenting itself as 2D surface for adherent cells to attach. In addition, multiple layers of mesh can we arranged in roller bottle, resulting in increases of total available surface area ranging from about 2 to 20 times that of a standard roller bottle. Thus, existing roller bottle facilities and processing, including cell seeding, media exchange, and cell harvesting, can be modified by the addition of the improved cell culture substrate disclosed herein, with minimal impact on existing operation infrastructure and processing steps.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The packed bed cell culture substrate of one or more embodiments can consist of the woven cell culture mesh substrate without any other form of cell culture substrate disposed in or interspersed with the cell culture substrate. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizonal or vertical axis.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a method of using a cell culture bioreactor for differentiating stem cells in situ in the bioreactor, the method comprising: providing a bioreactor vessel, the bioreactor vessel comprising: a cell culture chamber within the bioreactor vessel, the cell culture chamber comprising an inlet for flowing fluid into the cell culture chamber and an outlet for flowing fluid out of the cell culture chamber, and a cell substrate disposed in the cell culture chamber and configured to culture cells thereon. The method further comprising seeding undifferentiated stem cells onto the cell substrate in the cell culture chamber; and perfusing the cell culture chamber with a differentiation media configured to promote differentiation of the undifferentiated stem cells into a specified cell lineage, whereby the undifferentiated stem cells become differentiated cells.

Aspect 2 pertains to the method of Aspect 1, further comprising, after perfusing the cell culture chamber with the differentiation media, perfusing the cell culture chamber with a dissociation reagent configured to release the differentiated cells from the cell substrate.

Aspect 3 pertains to the method of Aspect 2, further comprising a harvesting step comprising removing the released differentiated cells from the cell culture chamber.

Aspect 4 pertains to the method of Aspect 1, further comprising, after perfusing the bioreactor with the differentiation media, washing the cell culture chamber with a wash solution.

Aspect 5 pertains to the method of Aspect 4, wherein the wash solution comprises a fluid media or a phosphate-buffered solution (PBS).

Aspect 6 pertains to the method of Aspect 2 or Aspect 3, further comprising, after washing the cell culture chamber with a wash solution, perfusing the cell culture chamber with a dissociation reagent configured to release the differentiated cells from the cell substrate.

Aspect 7 pertains to the method of Aspect 6, further comprising a harvesting step comprising removing the released differentiated cells from the cell culture chamber.

Aspect 8 pertains to the method of Aspects 1-7, further comprising: providing a coating solution for coating the cell substrate; and before seeding undifferentiated stem cells, inputting the coating solution into the cell culture chamber through the inlet such that the coating solution contacts the cell substrate to coat the cell substrate.

Aspect 9 pertains to the method of Aspect 8, further comprising removing an excess of the coating solution from the cell culture chamber via the outlet or the inlet, wherein, after removing the coating solution, a coated cell substrate remains in the cell culture chamber.

Aspect 10 pertains to the method of Aspect 8 or Aspect 9, further comprising washing the cell culture chamber with a washing solution during or after removing the coating solution.

Aspect 11 pertains to the method of Aspects 8-10, wherein providing the coating solution comprises preparing a coating solution suitable for a particular cell culture application or a particular cell type.

Aspect 12 pertains to the method of Aspects 8-11 wherein the coating solution comprises a material for enhancing cell attachment and/or growth on the cell substrate.

Aspect 13 pertains to the method of Aspects 8-12, wherein the coating solution comprises at least one of extracellular matrix proteins, fibronectin, collagen, a hydrogel solution, a polymer solution, and recombinant proteins.

Aspect 14 pertains to the method of Aspects 1-13, wherein the cell substrate comprises a first side, a second side opposite the first side, a thickness separating the first side and the second side, and a plurality of openings formed in the cell substrate and passing through the thickness of the cell substrate.

Aspect 15 pertains to the method of Aspects 1-14, wherein the cell substrate comprises at least one of a molded polymer lattice sheet, a 3D-printed lattice sheet, and a woven mesh sheet.

Aspect 16 pertains to the method of Aspects 1-15, wherein the cell substrate comprises a polymer material.

Aspect 17 pertains to the method of Aspect 16, wherein the polymer material is at least one of polystyrene, polyethylene terephthalate, polycarbonate, polyvinylpyrrolidone, polybutadiene, polyvinylchloride, polyethylene oxide, polypyrroles, and polypropylene oxide.

Aspect 18 pertains to the method of Aspects 1-17, wherein the bioreactor is configured to provide uniform fluid flow through the cell substrate and/or cell culture chamber.

Aspect 19 pertains to a system for culturing adherent cells in a bioreactor, the system comprising: a bioreactor vessel comprising a cell culture chamber within the bioreactor vessel, the cell culture chamber comprising an inlet for flowing fluid into the cell culture chamber and an outlet for flowing fluid out of the cell culture chamber, and a cell substrate disposed in the cell culture chamber and configured to culture cells thereon; a recirculation loop configured to supply fluid to the bioreactor vessel via the inlet and remove fluid from the bioreactor vessel via the outlet; and a differentiation media vessel configured to supply a differentiation media to the cell culture chamber for differentiating stem cells into a specified cell lineage, whereby the undifferentiated stem cells become differentiated cells.

Aspect 20 pertains to the system of Aspect 19, wherein the differentiation media vessel is fluidly connected to the cell culture chamber via the recirculation loop.

Aspect 21 pertains to the system of Aspect 19 or Apect 20, further comprising a cell inoculum source for inputting undifferentiated cells into the cell culture chamber.

Aspect 22 pertains to the system of Aspects 19-21, further comprising a coating solution vessel fluidly connected to the cell culture chamber and configured for holding a substrate coating solution.

Aspect 23 pertains to the system of Aspects 19-22, further comprising one or more pumps for circulating at least one of a cell culture media, a differentiation media, a washing solution, a dissociation regent, and a cell inoculum through the recirculation loop or into the cell culture chamber.

Aspect 24 pertains to the system of Aspect 23, further comprising a controller for controlling the one or more pumps.

Aspect 25 pertains to the system of Aspect 24, wherein the controlling of the one or more pumps comprises controlling a flow rate or a flow direction of fluid in the bioreactor system.

Aspect 26 pertains to the system of Aspect 24 or 25, wherein the controller comprises a processor and a memory, the memory containing instructions and being in communication with and readable by the processor.

Aspect 27 pertains to the system of Aspect 26, wherein, when the instructions are executed by the processor, the controller receives a signal for at least one of: injecting the undifferentiated cells in the cell culture chamber, injecting the differentiation media into the cell culture chamber, injecting the washing solution into the cell culture chamber, injecting the coating solution into the cell culture chamber, injecting a cell culture media into the cell culture chamber, and injecting the dissociation reagent into the cell culture chamber.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A method of using a cell culture bioreactor for differentiating stem cells in situ in the bioreactor, the method comprising:
providing a bioreactor vessel, the bioreactor vessel comprising:
a cell culture chamber within the bioreactor vessel, the cell culture chamber comprising an inlet for flowing fluid into the cell culture chamber and an outlet for flowing fluid out of the cell culture chamber, and
a cell substrate disposed in the cell culture chamber and configured to culture cells thereon;
seeding undifferentiated stem cells onto the cell substrate in the cell culture chamber; and
perfusing the cell culture chamber with a differentiation media configured to promote differentiation of the undifferentiated stem cells into a specified cell lineage, whereby the undifferentiated stem cells become differentiated cells.

2. The method of claim 1, further comprising, after perfusing the cell culture chamber with the differentiation media, perfusing the cell culture chamber with a dissociation reagent configured to release the differentiated cells from the cell substrate.

3. The method of claim 2, further comprising a harvesting step comprising removing the released differentiated cells from the cell culture chamber.

4. The method of claim 1, further comprising, after perfusing the bioreactor with the differentiation media, washing the cell culture chamber with a wash solution.

5. (canceled)

6. The method of claim 2, further comprising, after washing the cell culture chamber with a wash solution, perfusing the cell culture chamber with a dissociation reagent configured to release the differentiated cells from the cell substrate.

7. The method of claim 6, further comprising a harvesting step comprising removing the released differentiated cells from the cell culture chamber.

8. The method of claim 1, further comprising:
providing a coating solution for coating the cell substrate;
before seeding undifferentiated stem cells, inputting the coating solution into the cell culture chamber through the inlet such that the coating solution contacts the cell substrate to coat the cell substrate.

9. The method of claim 8, further comprising removing an excess of the coating solution from the cell culture chamber via the outlet or the inlet, wherein, after removing the coating solution, a coated cell substrate remains in the cell culture chamber.

10. The method of claim 8, further comprising washing the cell culture chamber with a washing solution during or after removing the coating solution.

11. The method of claim 8, wherein providing the coating solution comprises preparing a coating solution suitable for a particular cell culture application or a particular cell type.

12. The method of claim 8, wherein the coating solution comprises a material for enhancing cell attachment and/or growth on the cell substrate.

13-18. (canceled)

19. A system for culturing adherent cells in a bioreactor, the system comprising:
a bioreactor vessel comprising:
a cell culture chamber within the bioreactor vessel, the cell culture chamber comprising an inlet for flowing fluid into the cell culture chamber and an outlet for flowing fluid out of the cell culture chamber, and
a cell substrate disposed in the cell culture chamber and configured to culture cells thereon;
a recirculation loop configured to supply fluid to the bioreactor vessel via the inlet and remove fluid from the bioreactor vessel via the outlet; and
a differentiation media vessel configured to supply a differentiation media to the cell culture chamber for differentiating stem cells into a specified cell lineage, whereby the undifferentiated stem cells become differentiated cells.

20. The system of claim 19, wherein the differentiation media vessel is fluidly connected to the cell culture chamber via the recirculation loop.

21. The system of claim 19, further comprising a cell inoculum source for inputting undifferentiated cells into the cell culture chamber.

22. The system of claim 19, further comprising a coating solution vessel fluidly connected to the cell culture chamber and configured for holding a substrate coating solution.

23. The system of claim 19, further comprising one or more pumps for circulating at least one of a cell culture media, a differentiation media, a washing solution, a dissociation regent, and a cell inoculum through the recirculation loop or into the cell culture chamber.

24. The system of claim 23, further comprising a controller for controlling the one or more pumps.

25. The system of claim 24, wherein the controlling of the one or more pumps comprises controlling a flow rate or a flow direction of fluid in the bioreactor system.

26. The system of claim 24 or claim 25, wherein the controller comprises a processor and a memory, the memory containing instructions and being in communication with and readable by the processor.

27. The system of claim 26, wherein, when the instructions are executed by the processor, the controller receives a signal for at least one of: injecting the undifferentiated cells in the cell culture chamber, injecting the differentiation media into the cell culture chamber, injecting the washing solution into the cell culture chamber, injecting the coating solution into the cell culture chamber, injecting a cell culture media into the cell culture chamber, and injecting the dissociation reagent into the cell culture chamber.
